(19) United States
(12) Patent Application Publication
(10) Pub. No.: US 2019/0307372 A1
(43) Pub. Date: Oct. 10, 2019

(54) BATTERY-LESS SWEAT PATCH TO MEASURE BIOCHEMICAL COMPOSITION

(71) Applicants: Cesar A. OCAMPO, Santa Clara, CA (US); Hong Anh TRUONG, San Jose, CA (US); Cihan YILMAZ, Cambridge, MA (US)

(72) Inventors: Cesar A. OCAMPO, Santa Clara, CA (US); Hong Anh TRUONG, San Jose, CA (US); Cihan YILMAZ, Cambridge, MA (US)

(21) Appl. No.: 16/379,421

(22) Filed: Apr. 9, 2019

Related U.S. Application Data
(60) Provisional application No. 62/655,307, filed on Apr. 10, 2018.

(51) Int. Cl. A61B 5/145; A61B 5/1477; A61B 5/00

(52) U.S. Cl. CPC A61B 5/14517; A61B 5/1477; A61B 2560/0219; A61B 5/7435; A61B 5/7475; A61B 5/6833

(57) ABSTRACT

A wearable patch for measuring the biochemical composition of a fluid is disclosed. The wearable patch of the present disclosure may comprise a bonding layer configured to adhere to a subject's skin; a microfluidic chip comprising at least one inlet, a plurality of channels and at least one outlet; an electronic chip assembly comprising at least one sensor, the at least one sensor configured to align with the at least one outlet of the microfluidic chip; a wicking layer configured to move the sweat collected in the at least one outlet through the at least one sensor; and a protective layer.

### CROSS-REFERENCE TO RELATED APPLICATIONS

This application claims the benefit of U.S. Provisional Application No. 62/655,307 having a filing date of Apr. 10, 2018, which is incorporated by reference as if fully set forth.

### FIELD OF INVENTION

This invention relates generally to a device for measuring the biochemical composition of a fluid. More specifically, the present invention relates to a wearable patch for collecting sweat and measuring the biochemical composition thereof.

### BACKGROUND

Sweat contains a multitude of electrolytes and metabolites, such as lactate, glucose, protein and fatty acids. Measurements of certain electrolytes and metabolites may provide important insights into the health of a subject. For example, high lactate levels may be indicative of serious health conditions such as lactic acidosis and cardiac arrest. Other electrolytes and metabolites may provide information on other medical conditions, including, but not limited to, elevated glucose levels, nutritional deficiencies and ion imbalances. Therefore, measuring sweat may be an effective and noninvasive way to assess the health of a subject.

Sweat may be collected through a wearable patch that is adhered to the subject's skin via a bonding layer. One issue with collecting sweat through a wearable patch is the ability of the patch to capture all of the sweat from the skin without compromising the adhesiveness of the bonding layer. As such, it would be desirable to have a wearable patch that is capable of capturing sweat without reducing the adhesiveness of the bonding layer.

Further, it would be desirable to have a wearable patch that, in addition to collecting sweat, measures the biochemical composition of the collected sweat and moves the collected sweat out of the wearable patch.

### SUMMARY

A device and method for collecting sweat and measuring the biochemical composition thereof via a wearable patch is disclosed. The wearable patch may comprise a bonding layer, a microfluidics chip, at least one sensor, a wicking layer and a protective layer. The bonding layer is configured to bond the wearable patch to the subject's skin. The microfluidics chip is configured to capture and direct the sweat to the at least one sensor, which measures the biochemical composition of the sweat. The wicking layer is configured to move the sweat collected in the microfluidics chip through the at least one sensor. The wicking layer may be further configured to spread sweat across a surface area to better evaporate sweat out of the patch. The protective layer may be configured to allow vapor to flow out of the wearable patch while preventing other fluid from entering the system.

### BRIEF DESCRIPTION OF THE DRAWINGS

FIG. 1 is an exploded view of a wearable patch of the present disclosure;

FIG. 2A is an exploded view of a bonding layer and a corresponding liner of the wearable patch;

FIG. 2B is a top view of the bonding layer and the corresponding liner of the wearable patch;

FIG. 3A is a bottom view of a microfluidics chip of the wearable patch;

FIG. 3B is a top view of the microfluidics chip of the wearable patch;

FIG. 3C is a side view of the microfluidics chip of the wearable patch;

FIG. 4 is a top view of the electronic chip assembly of the wearable patch;

FIG. 5 is a top view of the wearable patch attached to the liner;

FIG. 6 is a depiction of a system comprising the wearable patch of the present disclosure and a user device;

FIG. 7 is a flowchart for manufacturing the wearable patch of the present disclosure; and

FIG. 8 is a flowchart for measuring the biochemical composition of sweat.

### DETAILED DESCRIPTION OF THE PREFERRED EMBODIMENTS

Examples of different patches, sensors and related components will be described more fully herein with reference to the accompanying drawings. These examples are not mutually exclusive, and features found in one example can be combined with features found in one or more other examples to achieve additional implementations. Accordingly, it will be understood that the examples shown in the accompanying drawings are provided for illustrative purposes only and they are not intended to limit the disclosure in any way. Like numbers refer to like elements throughout.

Below are described an apparatus and methods for collecting sweat and measuring the biochemical composition thereof via a wearable patch. The device and methods described may allow for real-time, non-invasive monitoring of a subject's health and fitness, among other applications. The device may be capable of collecting and measuring at least one substance found in sweat, including but not limited to, lactate, glucose, protein, electrolytes and fatty acids. The measured values of the at least one substance may be recorded and analyzed.

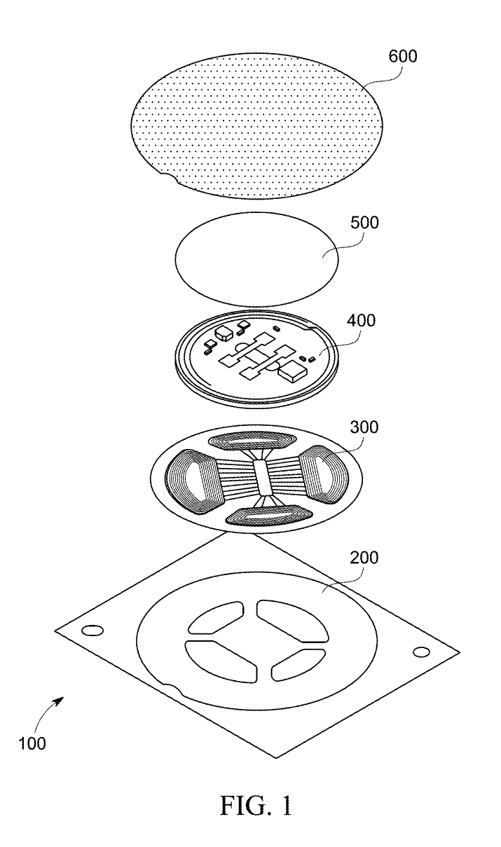

With reference to FIG. 1, an embodiment of the wearable patch 100 is illustrated. The wearable patch 100 of the present disclosure comprises a bonding layer 200, a microfluidics chip 300, an electronic chip assembly 400 comprising at least one sensor, a wicking layer 500 and a protective layer 600, as shown in FIG. 1. The wearable patch 100 may optionally comprise one or more supplemental bonding layers (not shown). The bonding layer 200 is configured to bond the wearable patch 100 to a surface. The surface may be the skin of a subject. Sweat emitted by the subject enters the microfluidics chip 300. The microfluidics chip 300 directs the sweat to the at least one sensor 407 located on the electronic chip assembly 400. The wicking layer may be configured to move the sweat collected in the at least one outlet through the at least one sensor 407 located on the electronic chip assembly 400. The wicking layer may be further configured to evaporate the sweat out of the wearable patch 100. The protective layer 600 protects the other layers of the wearable patch 100 from the surrounding environment.

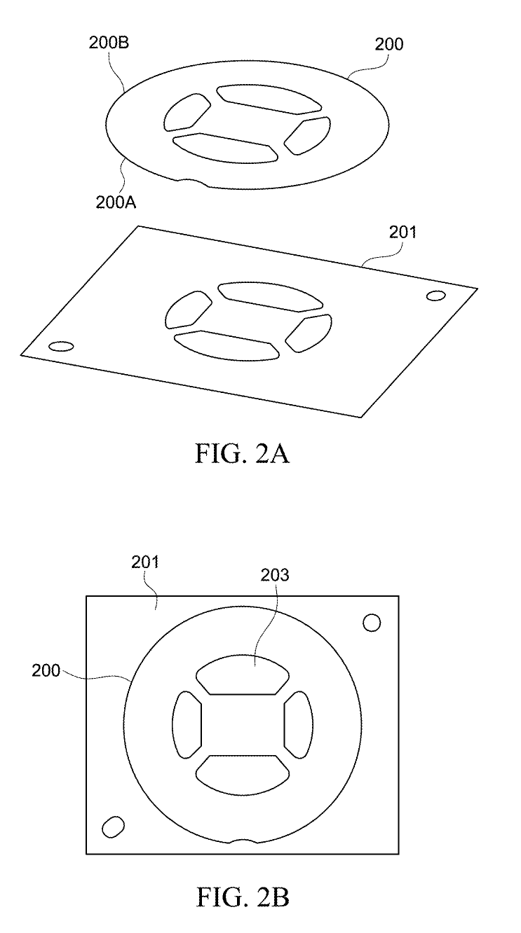

With reference to FIG. 2, the bonding layer 200 has a first portion 200A and a second portion 200B, a bonding material being distributed across the first portion 200A. The first portion 200A having the bonding material bonds the wearable patch 100 to a subject. The first portion 200A of the bonding layer 200 may be affixed to a liner 201 when it is not bonded to a subject, so as to preserve the bonding material distributed across the first portion 200A. The liner 201 may simply be peeled off, exposing the first portion of the adhesive layer 200 for bonding to a subject. In an embodiment, a bonding material may also be distributed across the second portion 200B of the bonding layer 200.

The bonding layer 200 may be comprised of a material that is both flexible and breathable. The use of a flexible material enables the bonding layer 200 to bend according to the contours of the subject's skin. The use of breathable material allows sweat to freely exit the skin. In an embodiment, the bonding layer 200 may comprise one of a single sided or double sided medical adhesive. However, as will be appreciated by one of ordinary skill in the art, the adhesive layer 200 may be comprised of any material capable of bonding to skin.

The bonding layer 200 has at least one opening 203 that allows sweat to enter the wearable patch 100 without affecting its chemical composition. The at least one opening 203 of the bonding layer 200 will be discussed in more detail below with respect to the microfluidics chip 300.

In some embodiments, the liner 201 may have at least one opening that aligns with the at least one opening 203 of the bonding layer 200. In alternative embodiments, the liner 201 does not have any openings.

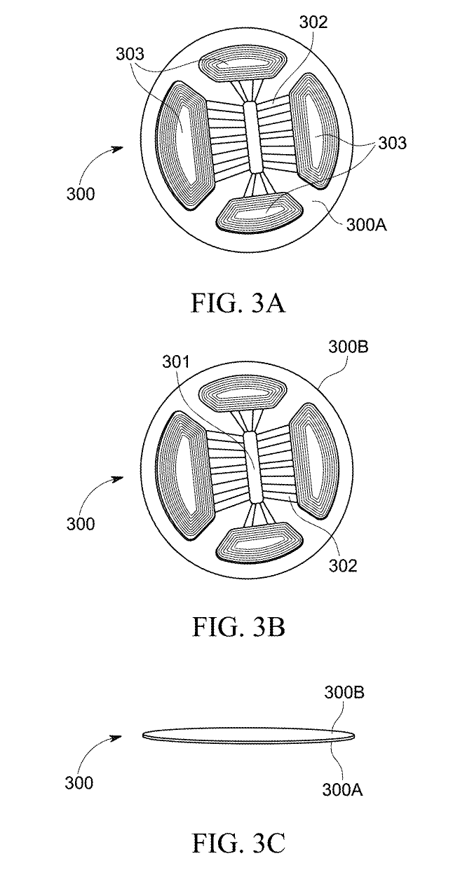

With reference to FIGS. 3A and 3B, an embodiment of the microfluidics chip 300 of the present disclosure is illustrated. The microfluidics chip 300 may comprise at least one inlet 303, at least one outlet 301 and a plurality of channels 302.

The microfluidics chip 300 may be comprised of a variety of different materials, including but not limited to glass, silicon, polymers or the like. For example, the microfluidics chip 300 may be comprised of one of polycarbonate, polyethylene terephthalate, polyvinylidene fluoride, polyvinylidene difluoride, polyurethane, cyclic olefin copolymer, cyclic olefin polymer, thermoplastic polyurethane, elastomers, gels or any organic or inorganic polymer. This list is meant to be illustrative and not exhaustive, and as will be appreciated by one of ordinary skill in the art, the microfluidics chip 300 may be comprised of various other materials.

The at least one inlet 303 may be an opening located on a first portion 300A of the microfluidics chip 300, as shown in FIG. 3A. The at least one outlet 301 may be an opening located on a second portion 300B of the microfluidics cup 300, as shown in FIG. 3B. The at least one inlet 303 may be configured to allow sweat to enter the wearable patch 100. In an embodiment, the at least one inlet 303 may be adjacent to the subject's skin when wearable patch 100 is bonded to the subject's skin, thereby enabling sweat to enter the wearable patch 100. In a further embodiment, the first portion 300A of the microfluidics chip may be adjacent to the second portion 200B of the bonding layer 200 and at least a portion of the at least one opening 203 of the bonding layer 200 may overlap with the at least one inlet 303 of the microfluidics chip 300. This allows the subject's sweat to enter the wearable patch 100. In a further embodiment, the at least one opening 203 of the bonding layer 200 may be substantially the same size and shape as the at least one inlet 303 of the microfluidics chip 300. This allows the subject's sweat to enter the wearable patch 100 without the chemical composition of the sweat being affected.

In an embodiment, the microfluidics chip 300 may comprise more than one inlet 303. For example, the microfluidics chip 300 illustrated in FIGS. 3A and 3B has four inlets 303. However, as will be appreciated by one of ordinary skill in the art, the microfluidics chip 300 may comprise a variety of different numbers of inlets 303.

In an embodiment, the at least one inlet 303 of the microfluidics chip 300 may comprise a curved shape with four sides. However, as will be appreciated by one of ordinary skill in the art, the at least one inlet 303 of the microfluidics chip 300 may be a variety of different shapes, including but not limited to, circular, semicircular, polygonal or elliptical.

The microfluidics chip further comprises a plurality of channels 302. Each channel of the plurality of channels 302 has a length, a width and a height. Each channel of the plurality of channels 302 may have a different length, width and/or height. Each channel of the plurality of channels 302 may be any geometry, including, but not limited to cylindrical, rectangular or the like. Further, each channel of the plurality of channels 302 may be straight or curved.

Each channel of the plurality of channels 302 may be any length. In an embodiment, each channel of the plurality of channels 302 may have a length that spans the distance from the at least one inlet 303 to the at least one outlet 301.

In an embodiment, each channel of the plurality of channels 302 may be large enough to allow molecules found in sweat to pass through the channel. In an embodiment, the channel may have a width that is greater than or equal to 200 nanometers. In a further embodiment, the channel may have a width that is greater than or equal to 200 nanometers and less than or equal to 500 micrometers.

In an embodiment, the height of each channel of the plurality of channels 302 may be greater than or equal to 10 microns. In a further embodiment, the height of each channel of the plurality of channels 302 may be greater than or equal to 10 microns and less than or equal to 2 millimeters.

The plurality of channels 302 may be coupled to both the at least one inlet 303 and the at least one outlet 301. In an embodiment, the plurality of channels may direct sweat from the at least on inlet 303 to the at least one outlet 301. The microfluidic chip 300 comprises a plurality of channels 302 to ensure fluid flow even if one channel of the plurality of channels 302 becomes clogged or pinched shut. Further, the plurality of channels 302 may comprise channels that go in different directions.

In an embodiment, the plurality of channels 302 may control the flow rate of the fluid from the at least one inlet 303 to the at least one outlet 301 by making adjustments to the channels through different mechanisms, including, but not limited to, plasma cleaning and chemical functionalization.

The plurality of channels 302 of the microfluidic chip may be at least one of capillary driven, pumped mechanically or pumped electrically. Therefore the plurality of channels 302 may be one of capillary driven, pumped mechanically or pumped electrically. The plurality of channels 302 may also be both capillary driven and pumped mechanically, capillary driven and chemically pumped or pumped mechanically and pumped electrically. In addition, the plurality of channels 302 may be capillary driven, pumped mechanically and pumped electrically.

In an embodiment, the at least one outlet 301 may be rectangular shaped with rounded edges. However, as will be appreciated by one of ordinary skill in the art, the at least one outlet 301 of the microfluidics chip 300 may be a variety of different shapes, including but not limited to, circular, semicircular, polygonal or elliptical.

The microfluidics chip may be manufactured using a variety of methods, including but not limited to 3D printing, micromolding, micromachining, roll to roll and lithography. This list is meant to be illustrative and not exhaustive, and as will be appreciated by one of ordinary skill in the art, the microfluidics chip 300 may be manufactured using other methods.

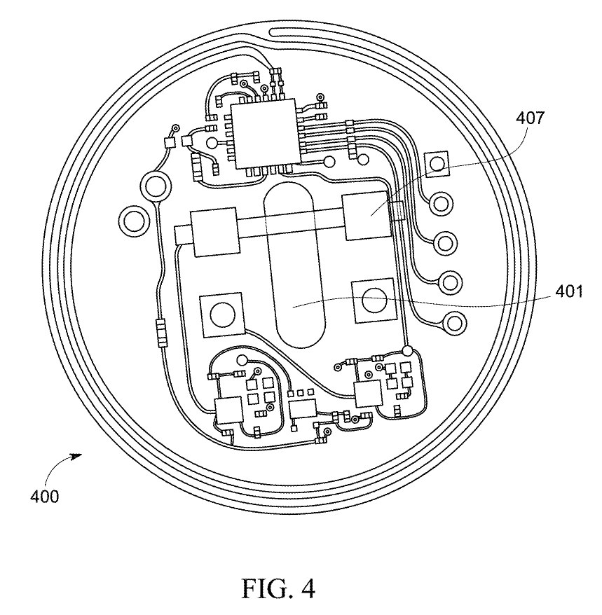

With reference to FIG. 4, the electronic chip assembly 400 comprises at least one sensor 407. In an embodiment, the electronic chip assembly 400 may be adjacent to the second portion of the microfluidics chip 300B comprising the at least one outlet 303. The electronic chip assembly 400 may comprise at least one aperture 401, at least a portion of which overlaps with the at least one outlet 303 of the microfluidic chip 300. This allows the sweat collected in the at least one outlet 303 of the microfluidics chip 300 to pass through the electronic chip assembly 400. In an embodiment, the at least one aperture 401 of the electronic chip assembly 400 may be substantially the same size and shape as the at least one outlet 303 of the microfluidic chip 300, and configured to align with the at least one outlet 303 of the microfluidic chip 300.

In an embodiment, the at least one aperture 401 of the electronic chip assembly 400 may be rectangular shaped with rounded edges. However, as will be appreciated by one of ordinary skill in the art, the at least one aperture 401 of the electronic chip assembly 400 may be a variety of different shapes, including but not limited to, circular, semicircular, polygonal or elliptical.

The at least one sensor 407 may overlap with at least a portion of the aperture 401 of the electronic chip assembly. This enables the sweat collected in the at least one outlet 301 of the microfluidic chip 300 to pass through the aperture 401 of the electronic chip assembly 400 and interact with the at least one sensor 407, thereby enabling the at least one sensor 407 to measure the biochemical composition of the collected sweat. In an embodiment, the at least one sensor 407 may span at least the width of the aperture.

The at least one sensor 407 may be a sensor for measuring biochemical composition. For example, the at least one sensor 407 may comprise one of a lactate sensor, a glucose sensor, an electrolyte sensor or a protein sensor. However, as will be appreciated by one of ordinary skill in the art, the at least one sensor 407 may comprise various other sensors for measuring biochemical composition.

The electronic chip assembly 400 may comprise more than one sensor 407. The sensors located on the electronic chip assembly 400 may be of the same or different type. For example, the electronic chip assembly 400 may comprise two lactate sensors or a lactate sensor and a glucose sensor. However, as will be appreciated by one of ordinary skill in the art, the electronic chip assembly 400 may comprise various combinations of sensors.

In an embodiment, the electronic chip assembly 400 may comprise printed circuit board assembly (PCBA). In a further embodiment, the electronic chip assembly 400 may comprise one of a rigid PCBA or a flex PCBA.

As will be appreciated by one of ordinary skill in the art, the electronic chip assembly 400 may have a protective coating configured to protect the electronics of the electronic chip assembly 400 from moisture and chemical contaminants. The protective coating may cover all of the surface area of the electronic chip assembly 400 except for the at least one sensor 407, so as to allow the least one sensor 407 to interact with and measure the collected sweat. In an embodiment, the protective coating may be a laminate. In an embodiment, the protective coating may be a conformal coating. Conformal coating conforms to the topology of the electronic chip assembly 400. The conformal coating may be a selective conformal coating. For example, in an embodiment, the electronic chip assembly 400 may have a conformal coating which covers the surface area of the electronic chip assembly 400, except for the at least one sensor 407, so as to allow the least one sensor 407 to interact with and measure the collected sweat.

The electronic chip assembly 400 may further comprise at least one communication protocol. The at least one communication protocol is for communicating with an external device, as will be discussed in more detail below.

Referring again to FIG. 1, a wicking layer 500 may be located adjacent to the electronic chip assembly 400. In an embodiment, the wicking layer 500 may be located above the electronic chip assembly 400.

The wicking layer 500 may be comprised of an absorbent material. In an embodiment the wicking layer 500 may be configured to pull the sweat collected in the at least one outlet 303 up and through the at least one sensor 407 located on the electronic chip assembly 400. In a further embodiment, the wicking layer 500 may spread the sweat across the surface of the at least one sensor 407, enabling the at least one sensor 407 to measure the biochemical composition of the sweat. Additionally, the wicking layer 500 may help evaporate the sweat out of the wearable patch 100 quickly, preventing sweat from pooling and exiting through the bottom of the patch, thereby compromising the integrity of the skin adhesive layer 201.

Referring again to FIG. 1, the protective layer 600 comprises an outer surface of the wearable patch 100. The protective layer 600 may overlay the wicking layer 500 and cover a sidewall of the wicking layer 500, the electronic chip assembly 400, the microfluidic chip 300 and the bottom adhesive layer 200. The protective layer 600 does not cover the at least one opening 203 of the bottom adhesive layer 200. The protective layer 600 may protect the other components of the wearable patch 100 from the surrounding environment.

In an embodiment, the protective layer 600 may be configured to allow vapor to flow out of the wearable patch 100. In a further embodiment, the protective layer may be impervious to fluid, thereby preventing fluid from entering the portions of the patch covered by protective layer 600.

In an embodiment, the protective layer 600 may be comprised of elastic nonwoven fabric material. The use of a nonwoven top adhesive layer allows sweat to evaporate out of the wearable patch. However, as will be appreciated by one of ordinary skill in the art, the protective layer 600 may be comprised of various materials.

The wearable patch 100 may further comprise an optional supplemental bonding layer (not shown). The optional supplemental bonding layer may be located between the microfluidic chip 300 and the electronic chip assembly 400. The optional supplemental bonding layer may have at least one opening that aligns with the at least one outlet 301 of the microfluidic chip 300 and the at least one aperture 401 of the electronic chip assembly 400, so as to allow the sweat collected in the at least one outlet 301 of the microfluidic chip 300 to pass through the supplemental bonding layer and the electronic chip assembly 400 and allow the at least one sensor 407 to interact with the collected sweat. The wearable patch 100 may further comprise additional bonding layers.

The optional supplemental bonding layer may be flexible and breathable. In an embodiment, the supplemental bonding layer may be one of a single sided or double sided medical adhesive. In an embodiment, the supplemental bonding layer may be the same material as the bonding layer 200.

In an embodiment, the bonding layer 200, the microfluidic chip 300, the electronic chip assembly 400 and the wicking layer 500 are circular shaped. However, as will be appreciated by one of ordinary skill in the art, the bonding layer 200, the microfluidic chip 300, the electronic chip assembly 400 and the wicking layer 500 may each comprise different shapes, including but not limited to rectangular, polygonal, elliptical or oval.

The wearable patch 100 may further comprise a battery (not shown) configured to supply power to the wearable patch 100. In an embodiment, the wearable patch 100 may further comprise a rechargeable battery. In an alternative embodiment, the wearable patch may further comprise a disposable battery. The battery may be various types, including but not limited to, coin cell and printed zinc.

The wearable patch 100 may also be powered externally. In an embodiment where the wearable patch 100 is powered externally, the cost of the wearable patch may be reduced, thereby making the wearable patch 100 more expendable. In an embodiment, the wearable patch 100 may be powered via near-field communication (NFC). In a further embodiment, the wearable patch 100 may be powered via passive NFC.

In an embodiment, the wearable patch 100 may be completely disposable. As such, the wearable patch 100 may be designed to be disposed of after a single use. For example, the wearable patch 100 may be comprised of inexpensive materials and may be powered externally. In an alternative embodiment, the wearable patch 100 may be partially disposable. As such, the wearable patch 100 may comprise some elements which are disposable and some which may be recycled for another use. For example, in an embodiment, the electronic chip assembly 400 may be designed for two or more uses and the protective layer 600 may be comprised of a material suitable for one use.

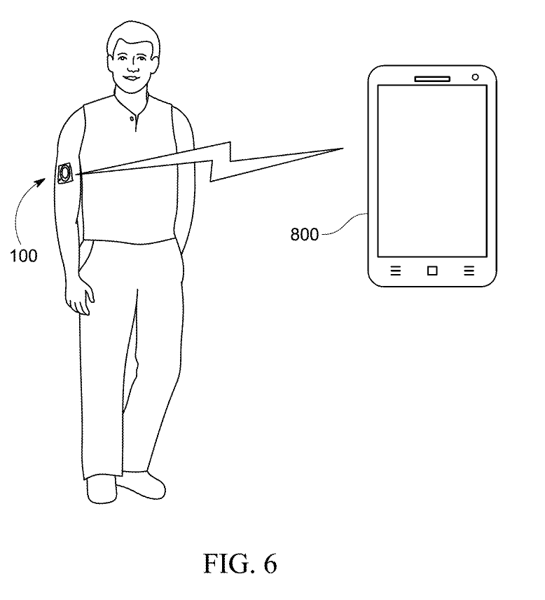

With reference to FIG. 6, the wearable patch 100 may be communicatively coupled to a user device 800. In an embodiment, the user device 800 may be a mobile device, such as a smart phone, tablet, portable computer, personal desktop computer or the like. In an embodiment, the wearable patch 100 may be communicatively coupled to the user device 800 wirelessly. For example, the wearable patch 100 may be communicatively coupled to the user device 800 through one of NFC, radio-frequency identification (RFID), Bluetooth, Wi-Fi, Bluetooth Low Energy (BLE) or Long-Term Evolution (LTE). These communication protocols are known in the art and are not discussed in detail here. As will be appreciated by a person having ordinary skill in the art, this list is meant to be illustrative and not exhaustive, and the wearable patch 100 may be communicatively coupled to the user device 800 through other wireless communication protocols.

In an embodiment, the wearable patch 100 may be configured to transmit the measurements of the at least one sensor 407 to the user device 800 and the user device 800 may be configured to receive the measurements of the at least one sensor 407.

In an embodiment, the user device 800 may be configured to run an application that collects the measurements of the at least one sensor 407. In a further embodiment, the user device 800 may have a graphical user interface for displaying the collected biochemical composition data. In a further embodiment, the user device 800 may be configured to format and display the biochemical composition data in a meaningful way on the graphical user interface. For example, the application may collect and format the biochemical composition data in a table, graph or chart.

In an embodiment, the application may analyze the collected biochemical composition data. For example, the application may be able to identify when a subject's lactic levels are above or below a certain threshold. In a further embodiment, the application may provide a signal indicating that a subject's lactic levels are above or below a certain threshold.

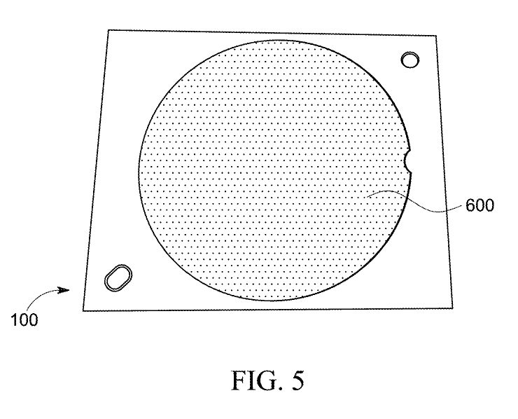

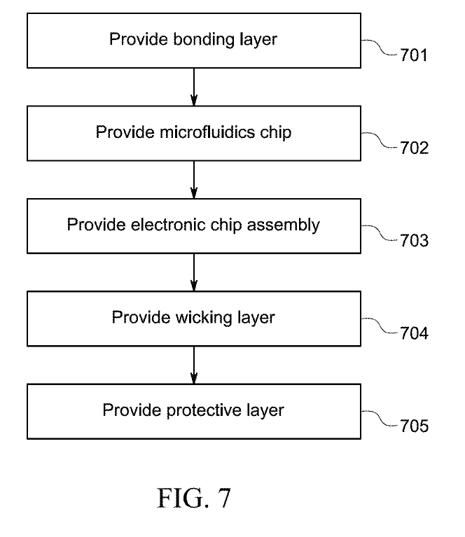

With reference to FIG. 7, a flowchart for manufacturing the wearable patch 1000 in accordance with the present disclosure is provided. At step 701, a bonding layer is provided. The bonding layer may be bonding layer 200 described with respect to FIGS. 1, 2A and 2B. At step 702, a microfluidics chip is provided. The microfluidics chip may be microfluidics chip 300 described with respect to FIGS. 1, 3A and 3B. At step 703, an electronic chip assembly is provided. The electronic chip assembly may be electronic chip assembly 400 described with respect to FIGS. 1 and 4. At step 704, a wicking layer is provided. The wicking layer may be wicking layer 500 described with respect to FIG. 1. At step 705, a protective layer is provided. The protective layer may be protective layer 600 described with respect to FIGS. 1 and 5. In an embodiment, the protective layer may overlay the bonding layer, the microfluidics chip, the electronic chip assembly and the wicking layer.

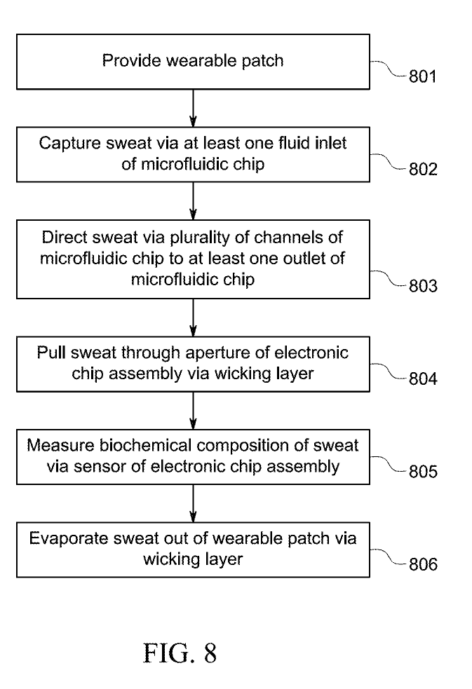

With reference to FIG. 8, a flowchart for measuring the biochemical composition of sweat in accordance with the present disclosure is provided. At step 801, a wearable patch in accordance with the present disclosure is provided. At step 802, the sweat of a subject is captured via the at least one inlet 303 of the microfluidic chip 300. At step 803, the sweat is directed from the at least one inlet 303 to at least one outlet 301 via the plurality of channels 302. If one channel of the plurality of channels 302 becomes pinched or clogged, the sweat may flow through any one of the other plurality of channels. Sweat is pooled in the at least one outlet 301 of the microfluidic chip 300. At step 804, the sweat collected in the at least one outlet 301 of the microfluidics chip 300 is moved through the at least one aperture 401 of the electronic chip assembly 400. This enables the sweat to come into contact with the at least one sensor 407 of the electronic chip assembly 400. At step 805, the biochemical composition of the sweat located in the at least one outlet 301 of the microfluidic chip 300 is measured via the at least one sensor 407 of the electronic chip assembly 400. The biochemical composition measurements may be transmitted to a user device for collection and analyzation. At step 805, the sweat is evaporated out of the wearable patch via the wicking layer.

Although features and elements are described above in particular combinations, one of ordinary skill in the art will appreciate that each feature or element can be used alone or in any combination with the other features and elements.

What is claimed is:

1. A device for measuring the biochemical composition of a fluid comprising:
a bonding layer;
a microfluidic chip having at least one inlet, a plurality of channels and at least one outlet;
an electronic chip assembly having at least one sensor, the at least one sensor configured to align with the at least one outlet of the microfluidic chip;
a wicking layer configured to move a fluid through the at least one sensor; and
a protective layer.

2. The device of claim 1, wherein the bonding layer is adjacent to the microfluidic chip and has at least one opening configured to align with the at least one inlet of the microfluidic chip.

3. The device of claim 2, wherein the electronic chip assembly is adjacent to the microfluidic chip and has at least one aperture configured to align with the at least one outlet of the microfluidic chip.

4. The device of claim 1, wherein the microfluidic chip is comprised of an organic or inorganic polymer.

5. The device of claim 1, wherein the microfluidic chip is manufactured by one of 3D printing, micromolding, micromachining, roll to roll or lithography.

6. The device of claim 1, wherein each channel of the plurality of channels of the microfluidic chip is configured to be at least one of capillary driven, pumped mechanically or pumped electrically.

7. The device of claim 1, wherein each channel of the plurality of channels of the microfluidic chip has a width, the width of each channel being greater than or equal to 200 nanometers and less than or equal to 500 micrometers.

8. The device of claim 1, wherein each channel of the plurality of channels of the microfluidic chip has a height, the height of each channel being greater than or equal to 10 microns and less than or equal to 2 millimeters.

9. The device of claim 1, wherein the protective layer is configured to allow vapor to flow out of the device.

10. The device of claim 1, wherein the electronic chip assembly is a printed circuit board assembly.

11. The device of claim 1, the device further comprising a battery configured to power the device.

12. The device of claim 1, wherein the wicking layer is located adjacent to the electronic chip assembly and configured to move sweat through the at least one sensor of the electronic chip assembly.

13. A system for measuring the biochemical composition of a fluid comprising:
a wearable patch comprising:
a bonding layer;
a microfluidic chip having at least one inlet, a plurality of channels, and at least one outlet;
an electronic chip assembly having at least one sensor, the at least one sensor configured to align with the at least one outlet of the microfluidic chip;
a wicking layer; and
a protective layer overlaying the wicking layer; and
a user device communicatively coupled to the wearable patch.

14. The system of claim 13, wherein the user device is a mobile device.

15. The device of claim 13, the wearable patch further comprising a battery configured to power the device.

16. The system of claim 13, wherein the wearable patch is powered via near-field communication.

17. The system of claim 13, wherein user device is communicatively coupled to the wearable patch through one of NFC, radio-frequency identification (RFID), Bluetooth, Wi-Fi, Bluetooth Low Energy (BLE) or Long-Term Evolution (LTE).

18. The system of claim 13, wherein the user device is configured to run an application that collects and analyzes the biochemical composition data of the sweat collected by the wearable patch.

19. The system of claim 18, wherein the user device comprises a graphical user interface configured to display the collected biochemical composition data.

20. The system of claim 19, wherein the user device is configured to format and display the biochemical composition data in a meaningful way on the graphical user interface.
